(19) United States
(12) Patent Application Publication
(10) Pub. No.: US 2026/0315181 A1
(43) Pub. Date: Oct. 15, 2026

(54) PATIENT TRACKER, MEDICAL SYSTEM AND TRACKING METHOD

(71) Applicant: B. Braun New Ventures GmbH, Freiburg im Breisgau (DE)

(72) Inventor: Jean Stawiaski, Freiburg (DE)

(21) Appl. No.: 19/465,185

(22) Filed: Jan. 30, 2026

(30) Foreign Application Priority Data
Feb. 4, 2025 (DE) 10 2025 104 086.9

(51) Int. Cl. A61B 90/00; A61B 34/10; A61B 34/20; A61B 34/30

(52) U.S. Cl. CPC A61B 90/39; A61B 34/30; A61B 2034/105; A61B 2034/2055; A61B 2034/2065; A61B 2090/3937; A61B 2090/397; A61B 2090/3995

(57) ABSTRACT

A hybrid patient tracker for optical tracking has an attachment section adapted to rigidly attach the patient tracker to a patient or patient attachment device. The patient tracker also has a plurality of infrared markers for tracking the patient tracker with an infrared camera. The patient tracker further has an optical pattern for tracking the patient tracker with a camera in the visible spectrum. The infrared markers and the optical pattern are arranged on oppositely-oriented sides of the patient tracker. The patient tracker can be part of a medical system and used in a tracking method.

### CROSS-REFERENCE TO RELATED APPLICATION

This application claims priority under 35 U.S.C. § 119 to German Application No. 10 2025 104 086.9, filed on Feb. 4, 2025, the content of which is incorporated by reference herein in its entirety.

### FIELD

The present disclosure relates to a hybrid patient tracker for optical tracking or a reference device that is adapted to be (preferably) firmly attached to a patient in order to reference the patient's position and/or orientation, for example, for a navigated surgical procedure. Furthermore, the disclosure relates to a medical system with such a patient tracker and a method for tracking such a patient tracker.

### BACKGROUND

In navigated surgery, trackers are typically used for surgical procedures on patients. At least one tracker is attached to the patient as a reference. The tracker is rigidly connected to the part of the body where the procedure is to take place so that (as far as possible) no relative movement occurs. For cranial surgery, a tracker can be attached directly to the skull or to a head clamp that secures the patient in place. A navigation system with a corresponding sensor can then detect the tracker and use it to track the patient, in this example the skull, i.e., to record (as far as possible) its current position and/or orientation. Such a tracker, which references or indicates the position and/or orientation of a patient, can be referred to as a patient tracker.

An instrument that also has a tracker can then be tracked relative to the patient tracker and thus the patient. For image-guided navigation, the patient is usually registered first, wherein captured 3D radiological image data (image data) of the patient is correlated with their current, real-time pose via the patient tracker. This is followed by the navigated use of surgical instruments on the patient.

In navigation, optical tracking using infrared radiation (IR) is the standard. Passive IR trackers have IR markers that reflect IR radiation. The reflection can be captured by an IR camera, which can then be used to determine the position and/or orientation of the tracker in real time. Active IR trackers, on the other hand, feature LEDs as IR markers, which themselves emit an IR signal that can be detected by the camera. In addition to optical tracking, there are other forms of position and/or orientation detection, such as electromagnetic tracking, e.g., using IMU (inertial measurement unit).

In cranial surgery in particular, it is often the case that the patient is placed in unusual positions compared to other surgical procedures in order to gain good access to the skull. For cranial procedures, the patient is typically in a semi-sitting position or prone position. These patient positions are challenging for conventional navigation systems, as visibility problems can often arise. An IR camera is usually unable to see or have in its field of view both the patient or their patient tracker and a surgical instrument such as a manipulator, e.g., a drill or a surgical microscope for intraoperative imaging. In this case, the surgical instrument cannot be tracked relative to the patient and navigated precisely.

In addition, the IR camera must be positioned specifically for patient registration, as the face is normally used for registration rather than the back of the head, since the face has more features. This step is particularly difficult and time-consuming, as it is not easy to find a position for the IR camera in which it can see both the patient tracker and a registration instrument (e.g., registration pointer) at the same time, while also allowing the registration instrument to reach the patient's face. This is particularly problematic when the patient is lying on their stomach. Since registration is usually followed by a navigated procedure on the back of the head in cranial surgery, typically to open the skull and create access to the brain, the IR camera usually has to be repositioned after registration, which took place on the face, in order to be able to capture instruments used on the back of the head.

### SUMMARY

In view of the problems described above, it is therefore a task of the invention to avoid or at least reduce the disadvantages, in particular to avoid, as far as possible, vision problems during registration and/or subsequent navigation, especially for cranial procedures, and to reduce the workload for medical personnel for navigated procedures.

This task is solved by a hybrid patient tracker according to claim 1 and a medical system and method according to the dependent claims. Advantageous embodiments are the subject of the dependent claims and/or described below in the description.

The hybrid patient tracker for optical tracking according to the disclosure has an attachment section that is adapted to rigidly attach the patient tracker to a patient or a patient attachment device, for example a head clamp, preferably without tools, a plurality of, preferably passive, infrared (IR) markers, preferably three or four, for tracking the patient tracker with/by means of an IR camera, and (at least) one optical pattern for tracking the patient tracker with/by means of a camera in the visible spectrum. The IR markers and the optical pattern are arranged on essentially opposite sides or on essentially opposite sections of the patient tracker.

A core idea of this disclosure is therefore to provide a hybrid patient tracker (hereinafter also referred to as a patient tracker or tracker) in which the infrared markers and the optical pattern are visible from essentially opposite directions. Therefore, the primary concern is not the (specific) application of the optical pattern (hereinafter also referred to as the pattern) and the IR markers, but rather the functionality described above. This has the advantage that the patient tracker can be visually detected from at least two (essentially) opposite directions, thus enabling significantly more positions for corresponding cameras. In addition, the patient tracker can be tracked both in the visible spectrum (light visible to humans) and in the IR spectrum, and can thus serve as a kind of connection or transmission element between an IR space, in which positions and/or orientations can be detected using IR, and a visible space or VIS space in which positions and/or orientations can be detected in the visible spectrum.

It should be understood that it is not necessary to attach the patient tracker directly to the patient in order to track the patient; rather, it is also possible to track the patient via the patient tracker using an intermediate device, which is itself attached to the patient as rigidly as possible (i.e., without any relative movement, or firmly). In other words, the attachment section is designed to connect the patient tracker rigidly, either directly or indirectly, to the patient.

The attachment section can be, for example, a screw or have a screw with which the hybrid patient tracker can be screwed onto the patient or a head clamp, for example. However, the disclosure is not limited to this; it may also involve, for example, a plug that can be inserted into a suitable receiving counterpart, e.g., a head clamp or a bone screw, as examples of the patient attachment device, or, for example, an adhesive section with which the hybrid patient tracker can be attached to the skull, or a headband that can be attached firmly and as non-slip as possible to a skull.

It should be noted at this point that a plurality means at least two. It should also be noted that, depending on the number of IR markers, which are typically spherical, the patient tracker can be tracked via the IR markers with varying degrees of freedom. Preferably, there are three or four IR markers, which enable spatial tracking in 6 degrees of freedom (e.g., 3 translational and 3 rotational), i.e., they specify the overall pose of the patient tracker. Four markers are often used for redundancy. However, the disclosure is not limited to this; there may also be fewer or more IR markers.

The IR markers are preferably passive and typically spherical. However, the disclosure is not limited to this; the IR markers can also be disc-shaped and/or active, for example.

IR markers can often be attached to and removed from trackers, for example for cleaning purposes or because they are often disposable products. It should therefore be noted that a corresponding disassembled hybrid patient tracker also falls within the scope of this disclosure, as does a patient tracker that is intended to be equipped with IR markers and an optical pattern in the manner disclosed.

It should be understood that the (optical) pattern is adapted for tracking in the visible spectrum (spectrum visible to humans). To this end, it may feature different sections in at least two different colors, e.g., black and white, or exhibit contrast. So these are explicitly not IR markers. The optical pattern may, for example, comprise lines (such as a barcode), squares (such as a QR code), and/or other shapes such as circles or ellipses, which may be arranged in a regular or irregular pattern.

The pattern is therefore adapted for tracking or locating the tracker, preferably spatially in 6D with three translational and three rotational degrees of freedom (6D tracking). However, the disclosure is not limited to this; it can also be adapted to 5D tracking, for example, if the pattern is rotationally symmetrical.

The pattern can, for example, be permanently attached to the frame of the hybrid patient tracker, for example by engraving or gluing. However, the disclosure is not limited to this; it may also be temporarily attached, for example, the patient tracker may include a plate or similar element that has the pattern and can be attached to and detached from a frame of the patient tracker, for example.

Preferably, the optical pattern is not visible from a direction from which the IR markers are visible, and vice versa. However, the disclosure is not limited to this. For example, the IR markers may also be visible from a direction from which the optical pattern is visible.

It should also be noted that a corresponding IR camera and a corresponding camera in the visual spectrum do not necessarily have to be aligned exactly opposite each other in order to track the patient tracker (simultaneously or sequentially). Their fields of view may also be offset in relation to each other, or their optical axes may have different directional components, and yet the tracker can still be tracked by both, as the IR markers and the optical pattern can still be visually detected up to a certain angle, even if the respective camera is looking at them at an angle. In other words, there can be many positions for an IR camera and a camera in the visual spectrum for tracking, but at least one in which they face each other.

The advantages of the disclosure are that the hybrid patient tracker can enable tracking from at least two substantially opposite directions. This makes it easier to avoid visibility problems during registration and subsequent navigation, for example by using two different cameras. In addition, the hybrid tracker enables tracking using an IR camera and a camera in the visual spectrum. This makes the tracker much more versatile for use with different optical sensors/cameras. This can be useful if, for example, a camera for the visual spectrum is required for imaging anyway, or if, for example, certain instruments are better suited to an optical pattern than IR markers, which are usually larger and spaced further apart, due to space constraints. Conversely, there are certain instruments on the market that are only available with IR markers, for example. Depending on which cameras are available or are needed to track other instruments, for example, the hybrid patient tracker can be tracked using IR markers or the optical pattern.

The hybrid patient tracker can also define, so to speak, an IR space in which instruments can be tracked relative to it using IR, and a second space for the visible spectrum in which instruments can be tracked relative to it in the visible spectrum. The hybrid patient tracker can thus divide the space into a first IR half and a second visible spectrum half for tracking purposes, and can itself lie between them and serve as a transmission or connection piece between these two halves, via which position and/or orientation data can be converted. So, when an IR camera and a visible spectrum camera are used, it is no longer necessary for them to see or track each other in order to know the transformation between an IR camera coordinate system and a visible spectrum camera coordinate system. they can also both track the hybrid patient tracker, so that data can be exchanged between the cameras via it, so to speak. This can be particularly useful if, as described above, the face is initially required for registration during cranial procedures, but the back of the head is subsequently required for navigation.

Preferably, the hybrid patient tracker has a frame or support structure to which the IR markers and the optical pattern are attached.

Preferably, the patient tracker and/or the carrier structure has at least a front side and a reverse side, with one of the IR markers and the optical pattern being arranged on the front side and the other being arranged on the reverse side. In other words, the IR markers are located on the front side and the optical pattern on the reverse side, or vice versa.

The front and reverse sides can be understood as two essentially opposite and/or parallel sides of the hybrid patient tracker, which in particular can divide the space, so to speak, into an IR tracking area or IR space (with the IR markers) and an area for tracking in the visible spectrum or visible space.

The hybrid patient tracker may also have a second optical pattern, which is preferably located on the same side (front or reverse side) as the (first) optical pattern. This allows, for example, redundancy for verification to be achieved and/or more angles or positions of a camera to be enabled for at least one of the optical patterns for tracking.

Furthermore, the disclosure relates to a medical system with a hybrid patient tracker, preferably as described above, and further with a first IR camera, which is set up to track the IR markers, and with a separate second camera, which is set up to track the optical pattern in the visible spectrum (i.e., to detect a position and/or orientation as close to real time as possible). Preferably, the first and second cameras are spaced apart from each other and/or can be positioned or moved individually.

It goes without saying that optical tracking is generally only possible if the target to be tracked (e.g., the IR markers) is within the field of view of the tracking camera (e.g., IR camera).

For simplicity, the second camera may also be referred to as a VIS camera (for Visible Spectrum) in the following. For simplicity, the first IR camera can also be referred to simply as the IR camera or the first camera.

In other words, the IR camera is set up to visually detect the IR markers and locate them (preferably all together) relative to the IR camera. A stereoscopic IR camera is preferred. Similarly, the VIS camera is designed to visually capture the optical pattern and record its position and/or orientation (as close to real time as possible) (relative to the VIS camera or in a VIS camera coordinate system). The VIS camera is preferably stereoscopic.

It should be noted that the first IR camera can also be an IR camera assembly consisting of several individual, possibly stereoscopic, IR cameras. The first IR camera can therefore also be a group of IR cameras. Accordingly, this group (as a whole) can be set up to track the IR markers. For this purpose, a transformation between the individual IR cameras of the first IR camera (group) is preferably known, e.g., because they track each other or each other jointly, or because they are immovable or fixedly connected to each other.

The same applies to the second camera. This can also be a group of cameras that is set up (as a whole) to track the optical pattern.

Preferably, the medical system further comprises a control unit.

Preferably, the control unit is aware of a relative transformation between the optical pattern and the IR markers of the hybrid patient tracker. For example, the distances between optical patterns and IR markers can be stored in the memory unit and made available to the control unit. Alternatively, the distances could be encoded in the optical pattern, for example.

Preferably, a control unit is set up using the relative transformation to the optical pattern relative position and/or orientation data, in particular of the VIS camera, the optical pattern, one or the instrument, and/or the 3D radiological image data, and to transfer relative position and/or orientation data relative to the IR markers, in particular of the IR camera, the IR markers, one or the instrument and/or the 3D radiological image data, into a common coordinate system.

In other words, the control unit is preferably adapted to determine position and/or orientation data (e.g., a position and/or orientation or coordinates, transformations, transformation matrices, quaternions, or the like) that are relative to the IR markers, in particular from the IR markers, of the hybrid tracker, and position and/or orientation data that are relative to the optical pattern, in particular the optical pattern, of the hybrid tracker, or have been determined, and to transform them into a common coordinate system.

The common coordinate system can be, for example, a coordinate system of the IR markers, the optical pattern, or a global coordinate system or an entire coordinate system of the hybrid tracker.

Preferably, the medical system has a memory unit that contains 3D radiological image data of a patient.

The control unit is preferably adapted, in particular for image-based and navigated surgical procedures on a patient, to spatially link 3D radiological image data (of the patient) with the hybrid patient tracker.

Preferably, a control unit is set up to spatially link the 3D radiological image data with the hybrid patient tracker via its (or by means of its) optical pattern.

Alternatively or additionally, and equally preferred, the control unit is adapted to spatially link 3D radiological image data with the hybrid patient tracker via its (or by means of its) IR markers.

The 3D radiological image data may have been obtained using MRI, CT, or ultrasound, for example, and normally represent at least the area of the patient's body where a navigated procedure is to be performed. It should be understood that the 3D (radiological) image data may also be available as individual 2D cross-sectional images, which together can form a three-dimensional image of the patient or the area of the body. The 3D image data therefore shows not only the patient's surface, but also (at least certain) internal anatomical structures.

It should be understood that, particularly for a navigated procedure, initial 3D radiological image data of a patient from the virtual space is typically correlated with the patient in the real space. This step is referred to as (patient) registration. A transformation between the patient and their 3D radiological image data is determined. The (current) (real) position and/or orientation of the patient is thus spatially linked to their 3D image data. However, since the position and/or orientation of the patient is recorded via the patient tracker as described above (both during registration and afterwards for navigation), the 3D image data is spatially linked to the patient tracker. If the patient moves and thus the patient tracker moves, the 3D radiological image data of the patient moves with them, preferably in such a way that it remains (as far as possible) virtually on the patient, so that the relative transformation between the patient and their 3D radiological image data (at least of an area of interest) does not change as far as possible. The spatial fixed link can therefore be understood as a static or constant transformation, or as meaning that a relative transformation between the hybrid patient tracker and the 3D radiological image data does not change over time and, preferably, the 3D radiological image data (as far as possible) has the same relative position virtually to the patient tracker as the patient has in reality. In other words, a fixed spatial link between the 3D image data and the hybrid patient tracker enables image-guided navigation.

It should be noted once again at this point that the position and/or orientation of the hybrid patient tracker is indicated by both the IR markers and the optical pattern, and that the hybrid patient tracker can therefore be tracked by both the IR camera and the VIS camera.

Accordingly, an instrument can also be tracked relative to the IR markers and also to the optical pattern of the hybrid patient tracker, in particular depending on where it is located (i.e., in the space detectable by the first or second camera) and/or whether it itself has an optical pattern or IR markers.

The control unit preferably has access to (all) data from the medical system, preferably in real time, preferably at least from the memory unit, the IR camera, and the VIS camera. The memory unit, IR camera, and VIS camera are therefore preferably set up to make their data available (digitally) to the control unit. For example, this means that when the IR camera tracks the IR markers of the hybrid patient tracker, i.e., determines a (preferably up-to-date) position and orientation, this position and/or orientation (data) is made available to the control unit (preferably in real time).

The control unit, the first IR camera, and the second camera (and optionally the memory unit) can be understood as a hybrid navigation system, which is suitable and/or adapted for tracking in the IR spectrum and visible spectrum.

Preferably, the medical system further comprises a medical instrument. The (medical) instrument can be, for example, a microscope, in particular a digital microscope for surgery, or a manipulator, e.g., a drill.

Preferably, the instrument itself has at least one IR marker, preferably three or four IR markers for spatial tracking in 3 rotational and 3 translational degrees of freedom, which can be detected by the IR camera and tracked relative to the IR markers of the hybrid patient tracker.

Alternatively or additionally, and equally preferably, the instrument itself has an optical pattern that can be captured by the VIS camera and tracked relative to the optical pattern of the hybrid patient tracker.

The control unit is particularly adapted to spatially link the 3D radiological image data with the hybrid patient tracker via its optical pattern; the instrument preferably has at least one IR marker, and the IR camera is adapted to track the instrument relative to the hybrid patient tracker via the IR marker of the instrument and the IR markers of the hybrid patient tracker relative to the hybrid patient tracker.

In other words, the control unit is preferably designed to perform patient registration (or the determination of the transformations for this purpose) with the aid of the VIS camera and the optical pattern of the hybrid tracker in the visible spectrum, so to speak, wherein the position and/or orientation of the optical pattern of the hybrid patient tracker is detected by the VIS camera, and then the instrument is preferably tracked (e.g., for a navigated procedure on the patient) using the IR camera relative to the hybrid patient tracker via the IR markers.

In other words, the VIS camera and the optical pattern of the hybrid patient tracker are initially preferred for registration in order to determine a position and/or orientation of the hybrid patient tracker via the optical pattern for the fixed link to the 3D radiological image data, and then the IR camera and IR markers of the hybrid patient tracker are used to determine a position and/or orientation of the hybrid patient tracker via the IR markers for relative tracking or navigation of the instrument to the hybrid patient tracker.

In other words, registration initially takes place in the visible space or spectrum, followed by navigation in the IR space or IR spectrum.

Alternatively or additionally, and also particularly preferred, the control unit is adapted to spatially link the 3D radiological image data with the hybrid patient tracker via its IR markers, the instrument has an optical pattern, and the second camera is adapted to track the instrument relative to the hybrid patient tracker via the optical pattern of the instrument and the optical pattern of the hybrid patient tracker.

In other words, the control unit is preferably set up to perform patient registration (or the determination of the transformations for this) with the aid of the IR camera and the IR markers of the hybrid tracker in the IR spectrum, so to speak, wherein the position and/or orientation of the IR markers of the hybrid patient tracker is detected by the IR camera, and then the instrument is preferably tracked (e.g., for a navigated procedure on the patient) using the VIS camera relative to the hybrid patient tracker via the optical patterns of the instrument and the hybrid patient tracker.

In other words, the IR camera and IR markers of the hybrid patient tracker are initially preferred for registration in order to determine a position and/or orientation of the hybrid patient tracker via the IR markers for fixed linking with the 3D radiological image data, and then the VIS camera and the optical pattern of the hybrid patient tracker are used to determine a position and/or orientation of the hybrid patient tracker via the optical pattern for relative tracking or navigation of the instrument to the hybrid patient tracker.

In other words, registration preferably takes place first in the IR space or spectrum, followed by navigation in the visible space or spectrum.

The medical system, or at least the control unit, the first camera, and the second camera, are therefore preferably adapted to perform patient registration using the IR markers of the hybrid patient tracker and to perform navigation or tracking of one or the instrument using the optical pattern of the hybrid patient tracker, or adapted to perform patient registration using the optical pattern of the hybrid patient tracker and navigation or tracking of one or the instrument using the IR markers of the hybrid patient tracker.

Preferably, the medical system has a display device, e.g., a monitor.

Preferably, the control unit is adapted to cause the display device to display a positionally accurate augmented reality representation of the instrument tracked relative to the hybrid patient tracker with the 3D radiological image data (as is known and customary in image-guided navigation).

Preferably, the first IR camera is mounted on a first, preferably mechanical arm, for positioning in the room. Mechanical does not mean electrical/robotic or for manual alignment/adjustment.

The VIS camera is preferably mounted on a second separate, preferably robotic, arm for positioning in space.

This has the advantage that the first IR camera and the second camera can be positioned independently of each other and can also be positioned in such a way that they can both track the hybrid patient tracker from two sides simultaneously.

They preferably have a common base, which is preferably a medical cart.

Preferably, the second camera is set up to create a 3D surface image of a patient, preferably of the patient's face, for image-based registration of the patient. In other words, the second camera is preferably set up to reconstruct a 3D surface of the patient, preferably the face, and to provide it, for example, as a point cloud. The 3D reconstruction can be based on photogrammetric and learning-based approaches, for example.

The control unit is preferably adapted to determine a transformation between the 3D surface image (of the patient) and the 3D radiological image data (of the patient), preferably by means of matching (e.g., “surface matching”).

In other words, the control unit is preferably set up to perform image or video registration (“video registration”) using 3D surface image.

Preferably, the second camera has a large field of view, in particular large enough to capture, for example, the patient's face together with the optical pattern of the hybrid tracker (when attached to the patient or the patient attachment device). Preference is given to the optical pattern captured with or in the patient's 3D surface image.

Preferably, the second camera is rigidly connected to IR markers, and preferably the first IR camera is set up to track the second camera spatially (in 6 degrees of freedom) via these IR markers. These IR markers can be active or passive.

The IR camera and VIS camera (optical) are calibrated as a priority.

Furthermore, the disclosure relates to a method for tracking a hybrid patient tracker of a medical system, preferably the system described above, comprising the following steps:
Tracking the IR markers of the hybrid patient tracker with the first IR camera; and
Tracking the optical pattern of the hybrid patient tracker with the second camera.

The steps can be performed sequentially or simultaneously.

The preferred method comprises the following steps:
Performing patient registration using the optical pattern of the hybrid patient tracker (and the second camera);
Tracking an instrument relative to the hybrid patient tracker using the IR markers of the hybrid patient tracker (and the first IR camera); and/or
Performing patient registration using the IR markers of the hybrid patient tracker (and the first IR camera);
Tracking an instrument relative to the hybrid patient tracker using the optical pattern of the hybrid patient tracker (and the second camera);

Preferably, the method further comprises at least one of the following steps, preferably the following steps:
Fixed spatial linking of the 3D radiological image data with the hybrid patient tracker via its optical pattern by the control unit;
Relative tracking of the instrument via at least one IR marker on the instrument and the IR markers on the hybrid patient tracker relative to the hybrid patient tracker by the first IR camera; and/or
Fixed spatial linking of the 3D radiological image data with the hybrid patient tracker via its IR markers by the control unit;
Relative tracking of the instrument via the optical pattern of the instrument and the optical pattern of the hybrid patient tracker relative to the hybrid patient tracker by the second camera.

Preferably, the control unit, the memory unit, the first IR camera, the second camera, and/or the medical system, as described above, are set up to perform at least one step of the method and/or the method.

According to an aspect that may be claimed independently, the disclosure therefore generally relates to a tracking method comprising the steps of:
Tracking IR markers of a hybrid patient tracker with a first IR camera; and
Tracking an optical pattern of the hybrid patient tracker with a second camera.

The preferred method (in general) comprises the following steps:

The preferred method comprises the following steps:
Performing patient registration using the optical pattern of the hybrid patient tracker (and the second camera);
Tracking an instrument relative to the hybrid patient tracker using the IR markers of the hybrid patient tracker (and the first IR camera); and/or
Performing patient registration using the IR markers of the hybrid patient tracker (and the first IR camera);
Tracking an instrument relative to the hybrid patient tracker using the optical pattern of the hybrid patient tracker (and the second camera);

The preferred method comprises the following steps:
Fixed spatial linking of 3D radiological image data with the hybrid patient tracker via its optical pattern by the control unit;
Relative tracking of an instrument with at least one IR marker via the at least one IR marker of the instrument and the IR markers of the hybrid patient tracker relative to the hybrid patient tracker by the first IR camera; and/or
Fixed spatial linking of the 3D radiological image data with the hybrid patient tracker via its IR markers by the control unit;
Relative tracking of the instrument via an optical pattern of the instrument and the optical pattern of the hybrid patient tracker relative to the hybrid patient tracker by the second camera.

Furthermore, the disclosure relates to a computer-readable storage medium, which may be claimed independently, comprising instructions that cause a computer (in particular a control unit, a first IR camera, a second camera, and/or a medical system, preferably as described above) to execute one of the methods or the method as described above.

### BRIEF DESCRIPTION OF THE DRAWINGS

The invention is explained in more detail below using preferred embodiments with the aid of figures:

FIG. 1 shows a front side of a hybrid patient tracker in a preferred embodiment according to the present disclosure;

FIG. 2 shows the reverse side of the hybrid patient tracker from FIG. 1;

FIG. 3 shows a medical system according to the present disclosure in an exemplary application on a patient;

FIG. 4 shows a medical system that is at least very similar to that shown in FIG. 3, in another example application on a patient;

FIG. 5 shows a method according to the present disclosure; and

FIG. 6 shows a preferred embodiment of the method shown in FIG. 5.

The figures are schematic in nature and are only intended to aid understanding of the invention. Identical elements are marked with the same reference signs. The features of the various embodiments can be interchanged.

### DETAILED DESCRIPTION

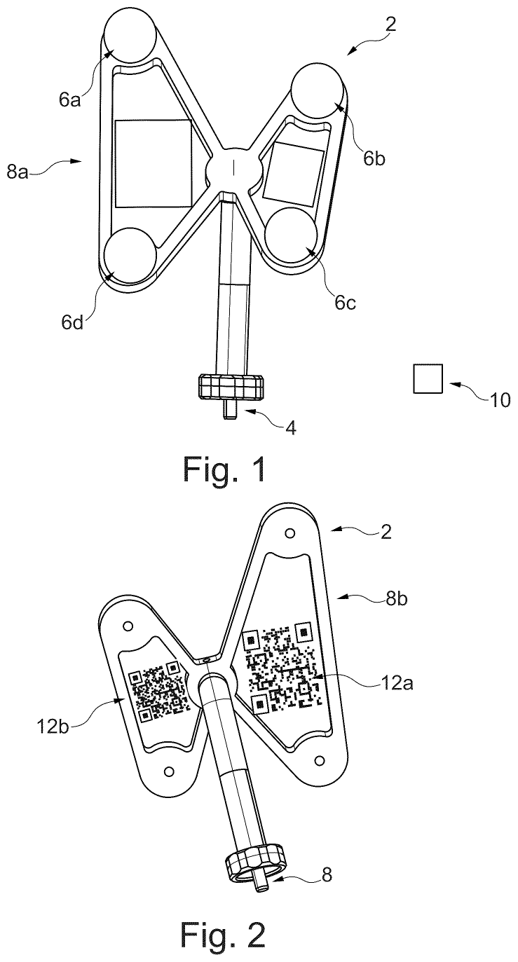

FIG. 1 shows a preferred embodiment of a hybrid patient tracker 2 for optical tracking. The hybrid patient tracker 2 has an attachment section 4 that is adapted to attach the patient tracker 2 rigidly, and preferably without tools, to a patient P or a patient attachment device 10. Furthermore, the patient tracker 2 has a plurality of infrared (IR) markers 6, which are passive here but can alternatively be active, for tracking the patient tracker 2 with an IR camera 16. Furthermore, the hybrid patient tracker 2 has at least one optical pattern 12 for tracking the patient tracker 2 with a camera 18 in the visible spectrum (not shown in FIG. 1, see FIG. 2). The IR markers 6 and the optical pattern 12 are arranged on essentially oppositely oriented sides 8 or on essentially opposite sections 8 of the patient tracker 2.

FIG. 1 shows a front side 8a, in this case a first section of the patient tracker 2. For simplicity, the side of the patient tracker 2 with the IR markers 6 is referred to as the front side 8a. The IR markers 6, in this case four pieces 6a through d, are arranged on the front side 8a.

FIG. 2 shows a corresponding reverse side 8b, in this case a second section of the patient tracker 2. In this case, the hybrid patient tracker 2 has two optical patterns 12a and 12b for tracking the patient tracker 2 with a camera 18 in the visible spectrum.

The IR markers 6 and, in this case, the two optical patterns 12a and 12b are therefore arranged in such a way that they are visible from two, at least essentially, opposite directions. The hybrid patient tracker 2 thus enables tracking from two (essentially) opposite directions, from a first direction in the IR spectrum and a second, (essentially) opposite direction in the visible spectrum.

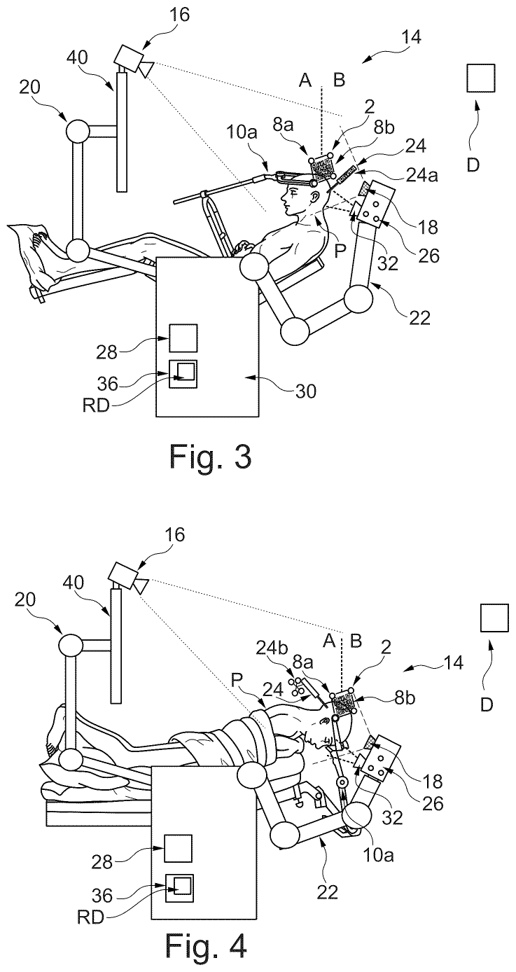

FIG. 3 shows a medical system 14 with the hybrid patient tracker 2 (shown schematically) from FIGS. 1 and 2. The medical system 14 further comprises a first IR camera 16 and a second camera 18.

The first IR camera 16 is set up to track the IR markers 6.

The second camera 18 is set up to track the two optical patterns 12a and 12b in the visible spectrum in this case. The second camera 18 can also be referred to as the VIS camera 18.

The patient attachment device 10 is here a head clamp 10a, to which the hybrid patient tracker 2 is attached by means of the attachment section 8. The head clamp 10 itself is attached to the skull of patient P.

For the sake of simplicity, only one optical pattern 12 is referred to in the following, but it should be understood that in this embodiment, this refers to the two optical patterns 12a and 12b.

The room is divided, so to speak, by the hybrid patient tracker 2 into a first IR area A and a second VIS area B, indicated by the dotted line. In the first IR range A, the IR markers of the hybrid patient tracker 2 are located and can therefore be tracked in the IR spectrum relative to the hybrid patient tracker 2 (by the IR camera 16). In the second VIS range B, the optical pattern 12 of the hybrid patient tracker 2 can be tracked there relative to the hybrid patient tracker 2 in the visible (VIS) spectrum (by the VIS camera 18).

The medical system 14 has a control unit 28.

Furthermore, the medical system 14 has a memory unit 36 with 3D radiological image data RD of the patient P, in this case of his skull.

Furthermore, the medical system 14 comprises an instrument 24. In this case, instrument 24 has (at least) one optical pattern 24a, and the VIS camera 18 is set up to visually detect and track it.

The control unit 28 is aware of a relative transformation RT (not shown) between the optical pattern 12 and the IR markers 6 of the hybrid patient tracker 2. The control unit 28 is designed to use the relative transformation RT to the optical pattern 12 to determine relative position and/or orientation data D, in particular of the second camera 18, the optical pattern 12, an instrument 24, and/or 3D radiological image data RD, and to determine relative position and/or orientation data D of the IR markers 6, in particular of the first IR camera 16, the IR markers 6, the instrument 24 and/or the 3D radiological image data RD, into a common coordinate system KOS.

This means that the control unit can convert coordinates, transformation matrices, etc. from the IR range to the VIS range. The hybrid patient tracker 2 thus serves as a transmission or connection element through which data from IR tracking and VIS tracking can be exchanged.

The control unit 28 is set up to spatially link the 3D radiological image data (RD) with the hybrid patient tracker 2 via its IR markers 6. (In particular) For this purpose, the IR camera 16 is set up to capture the most up-to-date (overall) pose of the IR markers 6 and to provide it to the control unit 28. This means that registration via the IR markers 6 of the hybrid tracker 2 is possible, in the IR range A, so to speak.

The second VIS camera is set up to track instrument 24 relative to hybrid patient tracker 2 via optical pattern 24a of instrument 24 and optical pattern 12 of hybrid patient tracker 2. In other words, this means that (after registration) navigation via the optical pattern 12 is possible, so to speak in the visible range B.

The first IR camera 16 can be positioned via a first, in this case mechanical, arm 20.

The second VIS camera 18 is attached to a second, separate arm 22, in this case a robotic arm, and can therefore be positioned separately from the IR camera. The second camera 18 can be moved from the first area A to the second area B with the help of the arm 22, so that it can see the face in area A and the back of the head of the patient P together with the optical pattern 12 of the hybrid patient tracker 2 in area B.

Both arms 20 and 22 have a common base 30, in this case a medical trolley 30a, for joint placement in the operating room.

Furthermore, the medical system 14 shows a microscope 32 for intraoperative, high-resolution images of patient P.

The second VIS camera 18 is set up to create a 3D surface image OA of patient P, preferably of patient P's face, for image-based registration of patient P, in particular in conjunction with the optical pattern 12 of the hybrid patient tracker 2. In other words, the field of view of the VIS camera 18 would be large enough to capture both the face of P and the optical pattern 12 of the hybrid tracker 2, but in this case, the face is on the other side of the hybrid tracker 2 than the optical pattern 12.

IR markers 26 are rigidly connected to the second camera 18. The first IR camera 16 is set up to track the second camera 18 via these IR markers 16 (if the second camera 18 is within the field of view of the first camera 16).

The medical system 14 also has a display device 40, in this case a monitor.

The fields of view of the respective cameras are shown schematically as dashed lines.

The following section explains some of the advantages of the hybrid patient tracker 2 and the medical system 14 using an application example in FIG. 3.

Patient P is in a semi-sitting position. This position is often used for procedures on the skull or back of the head.

First, image-based or video registration of patient P can be performed in IR range A. To do this, the second camera 18 is initially aligned with the patient's face in area A. The second camera 18 generates a 3D surface image OA of the patient's face P, which is registered by the control unit 28 with the radiological 3D images using surface matching. The 3D surface of patient P's face from the 3D surface image OA is registered with the 3D surface of patient P's face from the 3D radiological image data. The first camera 16 captures the pose of the second camera 18 relative to the hybrid patient tracker 2 via its IR markers 6 using the IR markers 26. This means that all necessary transformations between patient P (captured by the second camera 18) and his 3D radiological image data RD are known. During registration, the control unit 28 permanently links the 3D radiological image data RD to the hybrid patient tracker 2, so that when the patient P and thus the patient tracker 2 move later, the 3D radiological image data RD moves accordingly, so to speak, or its position can be adjusted accordingly so that a position or transformation between the instrument 24 and the 3D radiological image data of the actual position of the instrument 24 relative to the patient P (always) corresponds to image-based navigation. Initially, during registration, a transformation between the 3D radiological image data RD and the hybrid patient tracker 2 is determined and retained for subsequent navigation. Alternatively, registration could also be performed in the IR range A, for example, using a registration pointer that has its own IR markers.

After registration and for the actual procedure at the back of the head, the second camera 18 is moved to the position shown in FIG. 3. The instrument 24 is now navigated using the second camera 18 relative to the optical pattern 12 of the hybrid patient tracker 2, in visible space B, so to speak. The first camera 16 is no longer needed for navigation and there are no visibility problems.

The control unit 28 calculates a (possibly) up-to-date position of the instrument 24 relative to the 3D radiological image data RD, as it knows the transformation between the IR markers and the optical pattern 12. The control unit 28 causes the display device 40 to display a positionally accurate augmented reality representation of the 3D radiological image data with the instrument 24. Instrument 24 is therefore displayed in accordance with its actual position relative to the real patient P in relation to its 3D radiological image data. This allows, for example, a surgeon to see where the instrument 24 is located in relation to specific anatomical structures of patient P, particularly internal structures, which are depicted in the 3D radiological image data RD.

This means that there are no or fewer visibility problems and the workload for medical staff is significantly reduced.

FIG. 4 shows the medical system 14 from FIG. 3 or one that is at least very similar to it. The Instrument 24 has its own IR markers 24, in this case four for spatial tracking of the pose in 6 degrees of freedom (position & orientation). The IR camera 16 is set up to visually capture and track these, in particular relative to the IR markers 6 of the hybrid patient tracker 2. The instrument 24 may be a different instrument 24 or the same instrument 24 as shown in FIG. 3. This means that navigation of the instrument 24 relative to the hybrid patient tracker using its IR markers 6 is possible in the IR range A, so to speak.

Here, control unit 28 is additionally or alternatively adapted to spatially link the 3D radiological image data (RD) with the hybrid patient tracker 2 via its optical pattern 12. (In particular) For this purpose, the VIS camera 18 is set up to capture a (total) pose of the optical pattern 12 that is as up-to-date as possible and to provide it to the control unit 28. This means that registration via the optical pattern 12 of the hybrid tracker 2 is possible here, in the VIS range B, so to speak.

The following section explains some of the advantages of the hybrid patient tracker 2 and the medical system 14 using an example application in FIG. 4.

Patient P is lying in a prone position here. This position is also frequently used for procedures on the skull or back of the head.

Patient P is registered here in VIS area B. To do this, the VIS camera 18 is aligned with the face of patient P and a video recording is made. The VIS camera 18 takes a 3D surface image OA of the patient's face P, together with the optical pattern 12 of the hybrid patient tracker 2, so that both are present in the image, so to speak, meaning that a transformation between them is initially known. The control unit permanently links the 3D radiological image data RD to the hybrid patient tracker 12 via the optical pattern 12.

Alternatively, registration using a registration pointer, for example, would also be possible if this pointer had an optical pattern and was then tracked by the VIS camera 18 relative to the patient tracker 2. After registration, the VIS camera is no longer required.

The navigation of instrument 24 then takes place in IR range A. The IR camera tracks instrument 24 relative to the hybrid patient tracker 2 via its IR markers. The control unit transfers or transforms the (as far as possible) real-time, recorded position of the instrument 24 and the 3D radiological image data RD of the patient P into a common coordinate system, so that the realistic position of the instrument 24 in relation to the 3D radiological image data (corresponding to the position of the instrument 24 relative to the patient in reality) is known. The control unit causes the display device 40 to output a positionally accurate combined representation of the 3D radiological image data with the instrument 24.

This means that there are no or fewer visibility problems and the workload for medical staff is significantly reduced. Furthermore, the application examples in FIG. 3 and FIG. 4 illustrate the versatility and possible applications of the hybrid patient tracker 2.

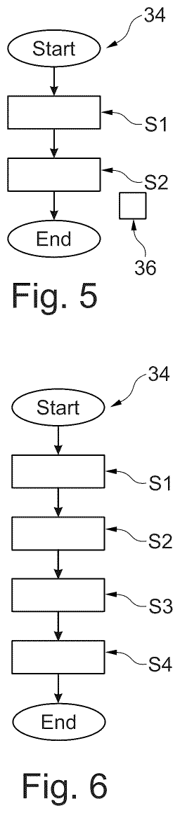

FIG. 5 shows a tracking method for a medical system 14, for example from FIG. 3 and/or FIG. 4, with the steps S, in particular performed simultaneously or sequentially:
S1: Tracking the IR markers 6 of the hybrid patient tracker 2 with the first IR camera 16; and
S2: Tracking the optical pattern 12 of the hybrid patient tracker 2 with the second camera 18.

Furthermore, FIG. 5 shows a computer-readable storage medium 36.

FIG. 6 shows the method from FIG. 5 with additional optional steps in two possible embodiments:
S3: Fixed spatial linking of the 3D radiological image data RD with the hybrid patient tracker 2 via its optical pattern 12 by the control unit 28;
S4: Relative tracking of the instrument 24 via the at least one IR marker 24b of the instrument 24 and the IR markers 6 of the hybrid patient tracker 2 relative to the hybrid patient tracker 2 by the first IR camera 16; or
S3: Fixed spatial linking of the 3D radiological image data RD with the hybrid patient tracker 2 via its IR markers 6 by the control unit 28;
S4: Relative tracking of the instrument 24 via the optical pattern 24a of the instrument 24 and the optical pattern 12 of the hybrid patient tracker 2 relative to the hybrid patient tracker 2 by the second camera 18.

### List of reference symbols

2 hybrid patient tracker
4 attachment section
P patient
6 IR Marker
8 side/section of the hybrid patient tracker 2
8a first side or front side
8b second side or reverse side
10 patient attachment device
10a head clamp
12 optical pattern
14 medical system
16 first IR camera
18 second camera in the visible spectrum or VIS camera
20 first arm
22 second arm
24 instrument
24a optical pattern instrument
24b IR marker instrument
26 IR marker for camera 18
28 control unit
RT relative transformation between IR markers 6 and optical pattern 12
D Position and/or orientation data
RD 3D radiological image data
KOS coordinate system
30 base
30a medical cart
32 microscope
34 method
36 computer-readable storage medium
38 memory unit
40 display device

1. A hybrid patient tracker for optical tracking, the hybrid patient tracker comprising:
an attachment section adapted to rigidly attach the hybrid patient tracker to a patient or a patient attachment device;
a plurality of infrared markers for tracking the hybrid patient tracker with an infrared camera; and
an optical pattern for tracking the hybrid patient tracker with a second camera in a visible spectrum,
the plurality of infrared markers and the optical pattern being arranged on oppositely-oriented sides of the hybrid patient tracker.

2. The hybrid patient tracker according to claim 1, wherein the hybrid patient tracker has at least one front side and at least one reverse side, and either:
the plurality of infrared markers is arranged on the at least one front side and the optical pattern is arranged on the at least one reverse side, or
the plurality of infrared markers is arranged on the at least one reverse side and the optical pattern is arranged on the at least one front side.

3. A medical system comprising:
the hybrid patient tracker according to claim 1;
an infrared camera; and
a second camera,
the infrared camera being adapted to track the plurality of infrared markers, and
the second camera being adapted to track the optical pattern in the visible spectrum.

4. The medical system according to claim 3, further comprising a control unit that is aware of a relative transformation between the optical pattern and the plurality of infrared markers, the control unit adapted to transfer relative position and/or orientation data to the optical pattern and relative position and/or orientation data to the plurality of infrared markers into a common coordinate system using the relative transformation.

5. The medical system according to claim 4, further comprising a memory unit that contains 3D radiological image data of the patient, wherein the control unit is adapted to spatially link the 3D radiological image data with the hybrid patient tracker via the optical pattern.

6. The medical system according to claim 5, wherein the control unit is adapted to spatially link the 3D radiological image data with the hybrid patient tracker via the plurality of infrared markers.

7. The medical system according to claim 3, further comprising an instrument with at least one infrared instrument marker, wherein the infrared camera is adapted to track the instrument relative to the hybrid patient tracker via the at least one infrared instrument marker and the plurality of infrared markers of the hybrid patient tracker.

8. The medical system according to claim 3, further comprising an instrument having an instrument optical pattern, wherein the second camera is adapted to track the instrument relative to the hybrid patient tracker via the instrument optical pattern and the optical pattern of the hybrid patient tracker.

9. The medical system according to claim 3, wherein the infrared camera is mounted on a first arm for positioning in space, and the second camera is mounted on a second arm for positioning in space.

10. The medical system according to claim 9, wherein the first arm is a mechanical arm.

11. The medical system according to claim 9, wherein the second arm is a robotic arm.

12. The medical system according to claim 9, wherein the first arm and the second arm have a common base.

13. The medical system according to claim 12, wherein the common base is a medical cart.

14. The medical system according to claim 3, wherein the second camera is adapted to create a 3D surface image of the patient for image-based registration of the patient.

15. The medical system according to claim 3, further comprising a plurality of infrared camera markers that are rigidly connected to the second camera, wherein the infrared camera is adapted to track the second camera via the plurality of infrared camera markers.

16. A method for tracking the hybrid patient tracker of the medical system according to claim 3, the method comprising the steps of:
tracking the plurality of infrared markers of the hybrid patient tracker with the infrared camera; and
tracking the optical pattern of the hybrid patient tracker with the second camera.

17. The method according to claim 16, wherein the step of tracking the plurality of infrared markers and the step of tracking the optical pattern are performed simultaneously.

18. The method according to claim 16, wherein the step of tracking the plurality of infrared markers and the step of tracking the optical pattern are performed sequentially.

19. The method according to claim 16, further comprising the steps of:
linking 3D radiological image data of the patient with the hybrid patient tracker via the optical pattern of the hybrid patient tracker; and
tracking an instrument relative to the hybrid patient tracker via at least one infrared marker on the instrument and the plurality of infrared markers of the hybrid patient tracker using the infrared camera.

20. The method according to claim 16, further comprising the steps of:
linking 3D radiological image data of the patient with the hybrid patient tracker via the plurality of infrared markers;
tracking an instrument relative to the hybrid patient tracker via an optical pattern on the instrument and the optical pattern of the hybrid patient tracker using the second camera.
